(19) United States
(12) Patent Application Publication
(10) Pub. No.: US 2025/0388826 A1
(43) Pub. Date: Dec. 25, 2025

(54) SYSTEMS AND PROCESS FOR THE PRODUCTION OF HYDROCARBON PRODUCTS

(71) Applicant: SABIC GLOBAL TECHNOLOGIES B.V., BERGEN OP ZOOM (NL)

(72) Inventors: Kenneth Francis LAWSON, Bangalore (IN); Mohammad Basheer AHMED, Bangalore (IN); Hatem Abdallah BELFADHEL, Bangalore (IN); Ahmad M. AL-SHEHRI, Bangalore (IN); Abdulrahman Shahid SARANG, Bangalore (IN)

(73) Assignee: SABIC GLOBAL TECHNOLOGIES B.V., BERGEN OP ZOOM (NL)

(21) Appl. No.: 18/881,262

(22) PCT Filed: Jul. 6, 2023
(86) PCT No.: PCT/EP2023/068755

§ 371 Date: Jan. 3, 2025

(30) Foreign Application Priority Data
Jul. 9, 2022 (IN) 202241039480

(51) Int. Cl. C10G 69/12

(52) U.S. Cl. CPC C10G 69/123; C10G 2300/1077; C10G 2300/4081

(57) ABSTRACT

Processes for the production of petroleum products from crude oil are disclosed. A process can include subjecting a vacuum resid stream, in a resid processing unit, to conditions suitable to produce pitch and hydrocarbons having a boiling temperature less than 450° C. Subjecting the pitch to conditions, in a pitch processing unit, to produce gaseous hydrocarbons, naphtha, distillate, and coke is also disclosed. The gaseous hydrocarbons, naphtha, distillate, and/or coke can be converted to other products such as ethylene, propylene, MTBE, and/or alkylates.

### CROSS REFERENCE TO RELATED APPLICATIONS

None.

### BACKGROUND OF THE INVENTION

### A. Field of the Invention

The invention generally concerns production of petrochemicals from a hydrocarbon feedstock (e.g., crude oil).

### B. Description of Related Art

Systems and processes to produce hydrocarbon products have been described. For example, U.S. Pat. No. 11,180,706 to Al-Sayed et al. describes a configuration for olefins production. The processes progressively separate a crude oil into light and heavy fractions, which can be upgraded using fixed bed hydroconversion unit, a fluidized catalytic conversion unit, or a residue hydrocracking unit. The upgraded fluids can be fed to a steam cracking unit for production of olefins. This process suffers from less than optimal performance, which can increase capital cost of the overall process and lower profitability.

Overall, while the technologies of producing olefins exist, they can be energy inefficient and expensive.

### SUMMARY OF THE INVENTION

A discovery has been made that provides a solution to at least one of the problems associated with production of hydrocarbon products from crude oil. In one aspect, the discovery can include processing crude oil to produce resid, which can be further processed to pitch. The pitch can be processed to produce any one of, any combination of, or all of coke, naphtha, distillate, and/or gaseous hydrocarbons. The gaseous hydrocarbons, naphtha, distillate, and/or coke can be converted to other products such as ethylene, propylene, MTBE, and/or alkylates. Use of the pitch in this manner can further increase the production efficiency of petrochemicals from crude oil.

In one aspect of the present invention, a process for the production of hydrocarbon products is described. The process can include (a) subjecting a vacuum resid stream, in a resid processing unit, to conditions suitable to produce pitch and hydrocarbons (e.g., hydrocarbons having a boiling temperature less than 450° C.). In step (b), the pitch can be subjected to conditions, in a pitch processing unit, to produce gaseous hydrocarbons, naphtha, distillate, and/or coke. From the pitch processing unit 6 to 10 wt. % of gaseous hydrocarbons, 18 to 22 wt. % naphtha, 33 to 65 wt. % distillate, and 10 to 35 wt. % of coke can be produced. In step (c), the gaseous hydrocarbons and the naphtha can be subjected to conditions suitable to produce ethylene, propylene, and/or C4 hydrocarbons. In step (d) at least a portion of the C4 hydrocarbons can be subjected to conditions to (i) produce, in a methyl tert-butyl ether (MTBE) production unit, MTBE and/or alkenes comprising 2-butenes, (ii) produce alkylates, in alkylates production unit, or (iii) a combination thereof. In some aspects, the vacuum resid is obtained from a crude oil process, which also produces light hydrocarbons and one or more distillate fractions. The light hydrocarbons can be separated to produce liquid petroleum gas (LPG) and/or the distillate fractions can be subjected to conditions to produce naphtha, gas oil, and atmospheric distillate. The LPG, naphtha, gas oil, and atmospheric distillate, or combination thereof can be provided to a steam cracking unit and subjected to conditions sufficient to produce additional ethylene, propylene and C4 hydrocarbons. The C4 hydrocarbons can be combined with the C4 hydrocarbons of step (c). The alkenes of step (d)(i) can be subjected to isomerization conditions suitable produce 1-butenes from the 2-butenes and residual butenes, and providing the 1-butenes to the MTBE production unit. The residual butenes can be subjected to hydrogenation conditions suitable to produce alkanes comprising butanes, and the alkanes can be provided to a steam cracking unit. In some aspects, a portion of the produced C4 hydrocarbons can be or are hydrogenated and then can be provided to the alkylates production unit, and optionally wherein at least a portion of the hydrogenated C4 hydrocarbons can be recycled to the steam cracking unit.

Other embodiments of the invention are discussed throughout this application. Any embodiment discussed with respect to one aspect of the invention applies to other aspects of the invention as well and vice versa. Each embodiment described herein is understood to be embodiments of the invention that are applicable to other aspects of the invention. It is contemplated that any embodiment or aspect discussed herein can be combined with other embodiments or aspects discussed herein and/or implemented with respect to any method or composition of the invention, and vice versa. Furthermore, compositions of the invention can be used to achieve methods of the invention.

The following includes definitions of various terms and phrases used throughout this specification.

The term “C# hydrocarbons”, wherein “#” is a positive integer, is meant to describe all hydrocarbons having # carbon atoms. Moreover, the term “C#+ hydrocarbons” is meant to describe all hydrocarbon molecules having # or more carbon atoms. Accordingly, the term “C2+ hydrocarbons” is meant to describe a mixture of hydrocarbons having 2 or more carbon atoms. The term “C2+ alkanes” accordingly relates to alkanes having 2 or more carbon atoms.

The terms “about” or “approximately” are defined as being close to as understood by one of ordinary skill in the art. In one non-limiting embodiment, the terms are defined to be within 10%, preferably within 5%, more preferably within 1%, and most preferably within 0.5%.

The terms “wt. %”, “vol. %”, or “mol. %” refers to a weight percentage of a component, a volume percentage of a component, or molar percentage of a component, respectively, based on the total weight, the total volume of material, or total moles, that includes the component. In a non-limiting example, 10 grams of component in 100 grams of the material is 10 wt. % of component.

The term “substantially” and its variations are defined to include ranges within 10%, within 5%, within 1%, or within 0.5%.

The terms “inhibiting” or “reducing” or “preventing” or “avoiding” or any variation of these terms, when used in the claims and/or the specification includes any measurable decrease or complete inhibition to achieve a desired result.

The term “effective,” as that term is used in the specification and/or claims, means adequate to accomplish a desired, expected, or intended result.

The use of the words “a” or “an” when used in conjunction with any of the terms “comprising,” “including,” “containing,” or “having” in the claims, or the specification, may mean “one,” but it is also consistent with the meaning of “one or more,” “at least one,” and “one or more than one.”

The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The systems and processes of the present invention can “comprise,” “consist essentially of,” or “consist of” particular ingredients, components, compositions, etc. disclosed throughout the specification. With respect to the transitional phrase “consisting essentially of,” in one non-limiting aspect, a basic and novel characteristic of the systems and processes of the present invention are their abilities to produce a variety of petrochemicals from crude oil and/or from pitch derived from crude oil.

Other objects, features and advantages of the present invention will become apparent from the following figures, detailed description, and examples. It should be understood, however, that the figures, detailed description, and examples, while indicating specific embodiments of the invention, are given by way of illustration only and are not meant to be limiting. Additionally, it is contemplated that changes and modifications within the spirit and scope of the invention will become apparent to those skilled in the art from this detailed description. In further embodiments, features from specific embodiments may be combined with features from other embodiments. For example, features from one embodiment may be combined with features from any of the other embodiments. In further embodiments, additional features may be added to the specific embodiments described herein.

### BRIEF DESCRIPTION OF THE DRAWINGS

Advantages of the present invention may become apparent to those skilled in the art with the benefit of the following detailed description and upon reference to the accompanying drawings.

FIG. 1 illustrates an embodiment of a system to produce hydrocarbon products from a crude feed that includes a crude oil processing unit coupled to a gaseous hydrocarbon separation unit, and a steam cracking unit coupled to the crude oil processing unit, and the gaseous hydrocarbon separation unit. The crude oil processing unit is also coupled to a reside processing unit, which is coupled to a pitch processing unit.

FIG. 2 is an illustration of the system of FIG. 1 with additional processing units shown.

FIG. 3 illustrates an embodiment of the system of FIG. 1 that includes a butene isomerization unit coupled to a hydrogenation unit that is coupled to the steam cracking unit.

FIG. 4 illustrates an embodiment of the system of FIG. 1 that includes an alkylation unit indirectly coupled to the steam cracking unit.

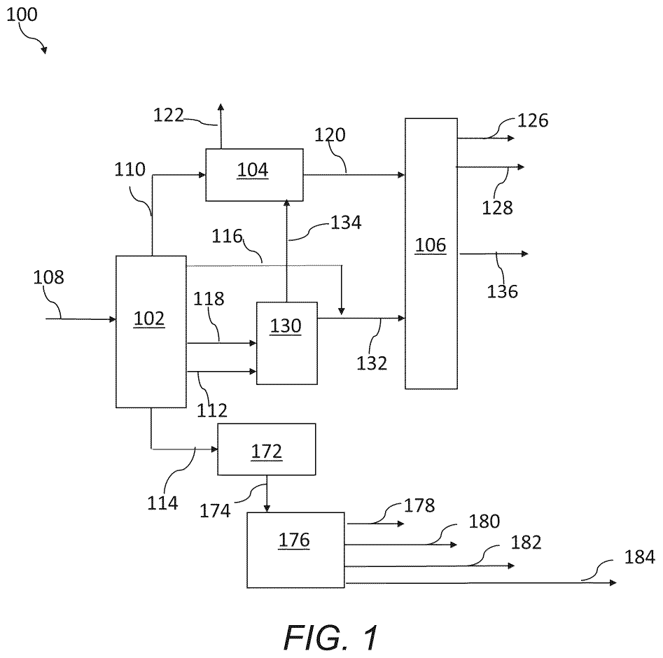

While the invention is susceptible to various modifications and alternative forms, specific embodiments thereof are shown by way of example in the drawings. The drawings may not be to scale. Still further, the schematics illustrated in FIGS. 1-4 can be combined with one another, which can be used, for example, to create a more robust process of producing a variety of petrochemical products from crude oil.

### DETAILED DESCRIPTION OF THE INVENTION

A discovery has been made that provides a solution to at least one of the problems associated with producing valuable hydrocarbon products from crude oil. In one aspect, the crude oil can be processed to produce resid, which can be further processed to pitch. The pitch can be processed to produce any one of, any combination of, or all of coke, naphtha, distillate, and/or gaseous hydrocarbons. These and other non-limiting aspects of the present invention are discussed in further detail in the following sections with reference to the Figures.

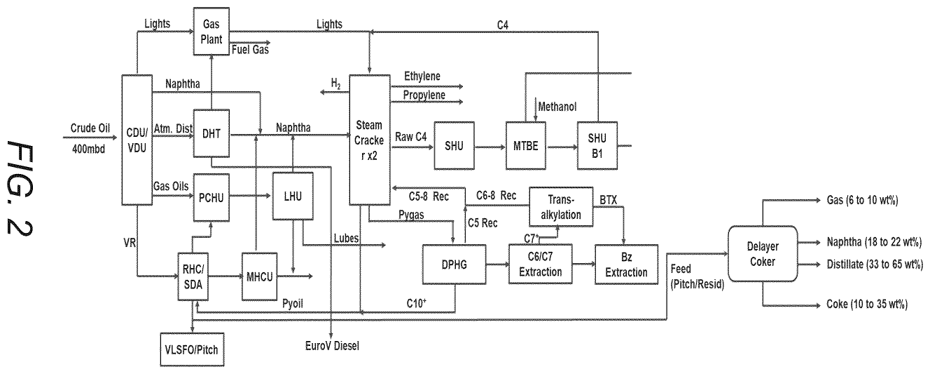

FIGS. 1 and 2 illustrate systems that produce valuable petroleum products from crude oil. Referring to FIG. 1, system 100 for producing petroleum products is described. FIG. 2 illustrates system 100 with various distillate processing units provided. System 100 can include a crude oil processing unit 102, a gaseous hydrocarbon separation unit 104, a steam cracking unit 106, a resid hydrocracking unit 172, and a pitch processing unit 176. Crude oil 108 enters feed separation 102. Crude oil can be the petroleum extracted from geologic formations in its unrefined form. The term crude oil can also include petroleum that has been subjected to water-oil separations and/or gas-oil separation and/or desalting and/or stabilization. Non-limiting examples of crude oil include Arabian Heavy, Arabian Light, other Gulf crudes, Brent, North Sea crudes, North and West African crudes, Indonesian, Chinese crudes, West Texas crude, and mixtures thereof, but also shale oil, tar sands, gas condensates and bio-based oils. The crude oil used as feed to the process of the present invention preferably is conventional petroleum having an API gravity of more than 20° API as measured by the ASTM D287 standard. In one aspect, the crude oil used in the process of the present invention is a light crude oil having an API gravity of more than 30° API. In another aspect, the crude oil used in the process of the present invention can include Arabian Light Crude Oil. Arabian Light Crude Oil typically has an API gravity of between 32-36° API and a sulfur content of between 1.5-4.5 wt. %.

In crude oil processing unit 102, at least one, two, three, four, or five hydrocarbon streams (e.g., light hydrocarbon stream 110, gas oil stream 112, resid stream 114, naphtha stream 116, and atmospheric distillate stream 118, in FIG. 1) can be produced under conditions previously described above for crude oil processing. (e.g., a temperature of 50° C. to 700° C.).

Vacuum resid stream 114 can exit crude oil processing unit 102 and enter resid hydrocracking unit 172. Resid hydrocracking unit 172 is capable of converting resid into pitch. Other hydrocarbon streams having can be produced in resid processing unit and be provided to distillate unit 130, combined with other streams, or transported to other processing units. Resid hydrocracking processes are well established. For example, three basic reactor types can be employed in commercial hydrocracking which are a fixed bed (trickle bed) reactor type, an ebullated bed reactor type and slurry (entrained flow) reactor type. Fixed bed resid hydrocracking processes are well-established and are capable of processing contaminated streams such as atmospheric residues and vacuum residues to produce the gas oil and naphtha. The catalysts used in fixed bed resid hydrocracking processes can include cobalt (CO), molybdenum (Mo), nickel (Ni), or a combination thereof on a refractory support, typically alumina. In case of highly contaminated feeds, the catalyst in fixed bed resid hydrocracking processes can also be replenished to a certain extend (moving bed). The process conditions can include a temperature of 350-450° C. and a pressure of 2-20 MPa gauge. Ebullated bed resid hydrocracking processes are also well-established and are inter alia characterized in that the catalyst is continuously replaced allowing the processing of highly contaminated feeds. The catalysts used in ebullated bed resid hydrocracking processes can include Co, Mo, Ni, or a combination thereof on a refractory support, typically alumina. The process conditions can include a temperature of 350-450° C. and a pressure of 5-25 MPa gauge. Slurry resid hydrocracking processes represent a combination of thermal cracking and catalytic hydrogenation to achieve high yields of distillable products from heavy resid feeds that are often highly contaminated. Such slurry resid hydrocracking processes are known (for example, U.S. Pat. No. 5,932,090, US 2012/0234726 A1 and WO 2014142874 A1). In the first liquid stage, thermal cracking and hydrocracking reactions can occur simultaneously in the bubble slurry phase at process conditions that include a temperature of 400-500° C. and a pressure of 15-25 MPa gauge. Resid, hydrogen and catalyst can be introduced at the bottom of the reactor and a bubble slurry phase can be formed; the height of which depends on flow rate and desired conversion. In these processes, catalyst can be continuously replaced to achieve consistent conversion levels through an operating cycle. The catalyst can be an unsupported metal sulfide that is generated in situ within the reactor. The heavy-distillate produced by resid upgrading can be recycled to the resid hydrocracking unit 172 until extinction.

Pitch stream 174 can exit resid hydrocracking unit 172 and enter pitch processing unit 176. In pitch processing unit 176, pitch can be converted into a light hydrocarbons, naphtha, distillate, and petroleum coke. An amount of light hydrocarbons produced can range from 6 to 10 wt. %. An amount of naphtha produced can range from 18 to 22 wt. %. An amount of distillate produced can range from 33% to 65 wt. %. An amount of coke produced can range from 10 to 35 wt. % The process in pitch processing unit 176 can thermally crack the long chain hydrocarbon molecules present in pitch stream 174 into shorter chain molecules. Light hydrocarbons can include C4 hydrocarbons, C3 hydrocarbons, C2 hydrocarbons, and/or methane, or combinations thereof. Light hydrocarbon stream 178, naphtha stream 180 (fourth naphtha stream), distillate stream 182 can be sent to steam cracking unit 106 to further the process. Light hydrocarbon stream 178 can also be sent to gaseous hydrocarbon separation unit 104. A combination of light hydrocarbon stream 178 and naphtha stream 180 can also be provided to gaseous separation unit 104. Depending on the type of distillate produced, distillate stream 182 can be provided to distillate processing unit 130 for further processing. Coke stream 184 can exit pitch processing unit 176 and be further processed, stored, or disposed.

Staying with FIG. 1, light hydrocarbon stream 110 can include C1-C4 hydrocarbons (e.g., ethane, propane, butanes, hydrogen and fuel gas) and can be provided to gaseous separation plant 104. In gaseous separation plant, fuel gas (e.g., methane) can be separated from the C1-C4 hydrocarbons to produce C2-C4 hydrocarbons. Any conventional method suitable for the separation of the gases may be employed in the context of the present invention. Accordingly, the gases can be subjected to multiple compression stages wherein acid gases such as CO₂ and H₂S may be removed between compression stages. In a following step, the gases produced can be partially condensed over stages of a cascade refrigeration system to about where only the hydrogen remains in the gaseous phase. The different hydrocarbon compounds may subsequently be separated by distillation. Fuel gas stream 122 can exit gaseous hydrocarbon separation unit 104 and be used in processing units as a source of fuel.

C2-C4 hydrocarbon stream 120 can exit gaseous hydrocarbon separation unit 104 and enter steam cracking unit 106. In steam cracking unit 106, the C2-C4 hydrocarbon feed can be subjected to steam cracking at a temperature of 600° C. to 900° C. (e.g., 600° C., 625° C., 650° C., 675° C., 700° C., 725° C., 750° C., 775° C., 850° C., 875° C., 900° C., or any value or range there between) and/or a pressure of 0.2 MPa to 0.3 MPa (e.g., 0.2 MPa, 0.21 MPa, 0.22 MPa, 0.23 MPa, 0.24 MPa, 0.25 MPa, 0.26 MPa, 0.27 MPa, 0.28 MPa, 0.30 MPa, or any value or range there between). At such a temperature and pressure the C2-C4 hydrocarbons are cracked to make ethylene and propylene. In a steam cracking process, the saturated hydrocarbons are broken down into smaller, often unsaturated, hydrocarbons such as ethylene and propylene by diluting the mixed hydrocarbon feed with steam and heating the mixture in a furnace in the absence presence of oxygen. The steam cracking reaction can have a residence times of 50-1000 milliseconds. Steam cracking unit can include one or more furnaces to process different compositions. For example, a furnace for C2-C4 hydrocarbons, and a furnace for naphtha. Steam cracking unit can have a fractionation unit (not shown) or a gas fractionation unit (not shown) capable of separating ethane and/or propane from the olefin product stream. Such fractionation units are well known in the art. Ethylene stream 126 can exit steam cracking unit 106, and be stored, sold, or used in other processing units. Propylene stream 128 can exit steam cracking unit 106, and be stored, sold, or used in other processing units.

Gas oil stream 112 and atmospheric distillate stream 118 can exit crude oil processing unit and enter distillate processing unit 130. In distillate processing unit 130, the steams can be further distilled and/or processed to remove impurities to form naphtha. FIG. 2 depicts a detailed illustration of one example of different distillation/purification units possible in a distillate processing unit. Light hydrocarbons stream 134 produced in the distillate processing unit can exit distillate processing unit 302 and enter gaseous separation unit 104 and be further processed. Naphtha stream 132 can exit the distillate processing unit and enter steam cracking unit 106. Other products can be produced in distillate processing unit 130 (e.g., diesel, lubricant oil, and the like). Distillate processing can include one or more fixed bed catalytic reactors with one or more fractionation units to separate desired products from unconverted material and can also incorporate the ability to recycle unconverted material to one or both of the reactors. Distillate processing reactors may be operated at a temperature of 200-600° C., preferably 300-400° C., a pressure of 3-35 MPa, preferably 5 to 20 MPa together with 5-20 wt. % of hydrogen (in relation to the hydrocarbon feedstock). Catalysts used in such processes comprise one or more elements selected from the group consisting of Pd, Rh, Ru, Ir, Os, Cu, Co, Ni, Pt, Fe, Zn, Ga, In, Mo, W and V in metallic or metal sulfide form supported on an acidic solid such as alumina, silica, alumina-silica and zeolites.

Steam cracking unit 106 can receive naphtha stream 132 and light hydrocarbons stream 120. In some aspects, naphtha stream 116 can be combined with naphtha stream 132 and/or directly provided to steam cracking unit 106. In steam cracking unit 106, naphtha stream 132 can be subjected to steam cracking conditions previously described to produce ethylene, propylene, and C4 hydrocarbons, Pygas and C7/8 hydrocarbons. Pygas can include aromatics, olefins, and paraffins ranging from C5s to C12s. The C4 hydrocarbons can be a mixture of butadiene, butane, and butenes (e.g., ethyl acetylene, vinyl acetylene, 1,3-butadiene, 1,2-butadiene, isobutylene, cis-2-butene, trans-2-butene, 1-butene, isobutane, and n-butane). Ethylene stream 126 can exit steam cracking unit 106 and be stored, transported, or used in other processing units. Propylene stream 128 can exit steam cracking unit 106 and be stored, transported, or used in other processing units. C4 hydrocarbon stream 136 can exit steam cracking unit 106 and be stored, transported, or used in other processing units.

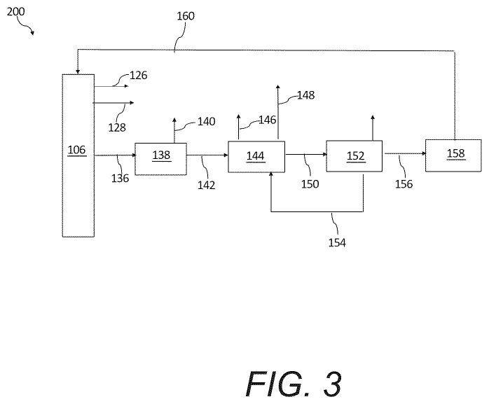

Referring to FIG. 3, in system 200, a portion or all of the C4 hydrocarbons produced from steam cracking unit 106 can be further processed produce methyl tert-butyl ether (MTBE) and additional C4 hydrocarbons. In some embodiment, the C4 hydrocarbons are produced from steam cracking unit 106 or different steam cracking units. C4 hydrocarbons stream 136 can exit steam cracking unit 106 and enter butadiene separation unit 138. In butadiene separation unit 138, butadiene can be separated from the C4 hydrocarbon to form a butadiene composition and a butene/butane composition. Butadiene stream 140 can exit butadiene separation unit 138 and be stored, transported or used in other processing units. Butene/butane stream 142 can exit butadiene separation unit 138 and enter methyl t-butyl ether (MTBE) production unit 144. In MTBE production unit 144, butene/butane stream 142 is contacted with methanol under conditions suitable to produce MTBE, a MTBE effluent, and 1-butene. MTBE stream 146 can exit MTBE production unit 140 and be stored and/or transported. 1-butene stream 148 can exit MTBE production unit 140 and be stored, used in other processing units and/or transported. MTBE effluent 150 can exit MTBE production unit 144 and enter butene isomerization unit 152. MTBE effluent can be a enriched 2-butene stream that includes 1-butene. In butene isomerization unit 152, 2-butene is contacted with a catalyst under isomerization conditions to produce a stream enriched in 1-butene and a residual butenes stream. The catalyst can by any known 2-butene isomerization catalyst. Isomerization conditions include reaction temperatures generally in the range of about 50° to 300° C. Reactor operating pressures usually can range from about atmospheric to 5 MPa. The amount of catalyst in the reactors can provide an overall weight hourly space velocity of from about 0.5 to 100 hr⁻¹. Enriched 1-butene stream 154 can exit butene isomerization unit 152 and enter MTBE production unit 144. Residual alkenes stream 156 can exit butene isomerization unit and enter C4 alkenes hydrogenation unit 158. In C4 alkenes hydrogenation unit 158, C4 alkenes can be contacted with a catalyst and hydrogen under conditions sufficient to produce additional C4 hydrocarbon and can optionally be recycled back to the steam cracking unit 106.

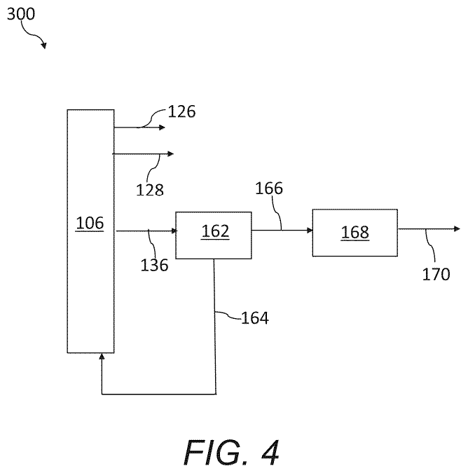

Referring to FIG. 4, in system 300, a portion or all of the C4 hydrocarbons produced from steam cracking unit 106 can be further processed to produce alkylates. In some embodiment, the C4 hydrocarbons are produced from steam cracking unit 106 or different steam cracking units. In system 300, a portion or all of C4 hydrocarbons stream 136 can exit steam cracking unit 106 and enter SHU (selective hydrogenation unit) 162. In SHU 162, the C4 hydrocarbons are subjected to conditions suitable to remove alkynes and/or dienes and produce an alkene composition and additional C4 hydrocarbons. Additional C4 hydrocarbons 164 can exit SHU 162 and enter steam cracking unit 106 to continue the process. Alkene composition stream 166 can enter alkylation unit 168. In alkylation unit 168, the alkene composition is subjected to conditions to produce alkylates. Alkylate stream 170 can exit alkylation unit 168 and be stored, transported, or processed in other units. Any portion of or all portions of FIGS. 1 and 2 can be combined with any portion of or all portions of FIGS. 3 and/or 4, and vice versa.

Although embodiments of the present application and their advantages have been described in detail, it should be understood that various changes, substitutions and alterations can be made herein without departing from the spirit and scope of the embodiments as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the above disclosure, processes, machines, manufacture, compositions of matter, means, methods, or steps, presently existing or later to be developed that perform substantially the same function or achieve substantially the same result as the corresponding embodiments described herein can be utilized. Accordingly, the appended claims are intended to include within their scope such processes, machines, manufacture, compositions of matter, means, methods, or steps.

1. A process for the production of hydrocarbon products, the process comprising:
(a) subjecting a vacuum resid stream, in a resid processing unit, to conditions suitable to produce pitch and hydrocarbons having a boiling temperature less than 450° C.; and
(b) subjecting the pitch to conditions, in a pitch processing unit, to produce gaseous hydrocarbons, naphtha, distillate, and coke.

2. The process of claim 1, further comprising:
(c) subjecting the gaseous hydrocarbons and the naphtha to conditions suitable to produce ethylene, propylene, and C4 hydrocarbons; and
(d) subjecting a least a portion of the C4 hydrocarbons to conditions to (i) produce, in a methyl tert-butyl ether (MTBE) production unit, MTBE and alkenes comprising 2-butenes, (ii) produce, in an alkylation production unit, alkylates, or (iii) a combination thereof.

3. The process of claim 1, wherein 6 to 10 wt. % of gaseous hydrocarbons, 18 to 22 wt. % naphtha, 33 to 65 wt. % distillate, and 10 to 35 wt. % of coke are produced.

4. The process of claim 2, further comprising obtaining the resid from crude oil processing that further produces light hydrocarbon and one or more distillate fractions.

5. The process of claim 4, further comprising separating the light hydrocarbons to produce liquid petroleum gas (LPG).

6. The process of claim 5, further comprising subjecting the distillate fractions to conditions to produce naphtha, gas oil, and atmospheric distillate.

7. The process of claim 6, further comprising providing the LPG, naphtha, gas oil, and atmospheric distillate to a steam cracking unit.

8. The process of claim 7, further comprising subjecting the naphtha, gas oil, atmospheric distillate, LPG, or a combination thereof to conditions sufficient to produce additional ethylene, propylene, and C4 hydrocarbons.

9. The process of claim 8, wherein the C4 hydrocarbons are combined with the C4 hydrocarbons of step (c).

10. The process of claim 2, wherein the alkenes are subjected to isomerization conditions suitable produce 1-butenes from the 2-butenes and residual butenes, and providing the 1-butenes to the MTBE production unit.

11. The process of claim 10, wherein the residual butenes are subjected to hydrogenation conditions suitable to produce alkanes comprising butanes, and the alkanes are provided to a steam cracking unit.

12. The process of claim 1, wherein a portion of the produced C4 hydrocarbons are hydrogenated and then are provided to an alkylation process, and optionally wherein at least a portion of the hydrogenated C4 hydrocarbons are recycled to a steam cracking unit.
